(19) United States
(12) Patent Application Publication
(10) Pub. No.: US 2009/0157002 A1
(43) Pub. Date: Jun. 18, 2009

(54) CATHETER HAVING COMMUNICATING LUMENS

(75) Inventors: John A. Dumot, Chagrin Falls, OH (US); Timothy E. Askew, Baltimore, MD (US); Jennifer Cartledge, Clemson, SC (US)

Correspondence Address: CONNOLLY BOVE LODGE & HUTZ LLP, 1875 EYE STREET, N.W., SUITE 1100, WASHINGTON, DC, 20006, US

(73) Assignee: CSA MEDICAL, INC., Baltimore, MD (US)

(21) Appl. No.: 11/956,890

(22) Filed: Dec. 14, 2007

(51) Int. Cl. A61M 1/00; A61M 25/00; A61B 18/02

(52) U.S. Cl. 604/131; 604/523; 604/265; 606/21

(57) ABSTRACT

A catheter having a plurality of longitudinal lumens for removing biological, natural and/or man-made materials from cavities, ducts, vessels, or other locations in a patient's body. The multi-lumen catheter comprises a longitudinally-extending suction lumen with suction holes through which materials pass into the lumen in response to suction forces generated by a source of negative pressure coupled to a proximal end of the lumen. A longitudinally-extending vent lumen coupled to a source of at least neutral vent pressure through, for example, an opening to ambient air at the proximal end of the lumen, and preferably, through vent holes disposed along a length of the catheter. A dividing septum between the adjacent lumens has one or more ports fluidically coupling the lumens. The ratio of the area of the suction holes and ports is such that the suction force at unobstructed suction holes is maintained below a desired maximum force for a given negative pressure when none or more of the suctions holes are obstructed. When a suction hole obstruction occurs, fluid is drawn into the suction lumen through the communication port(s). This compensating fluid flow prevents the suction forces from exceeding a predetermined maximum value during use even when one or more suction holes become obstructed. This maximum force may be set, for example, to avoid hematoma, to permit repositioning of the catheter during use, etc. thereby allowing for continuous suction.

### BACKGROUND

1. Field of the Invention

The present invention relates generally to catheters, and more particularly, to a catheter having communicating lumens.

2. Related Art

A catheter is a flexible tube made of latex, silicone, or Teflon that can be inserted into the body creating a channel for the passage of fluid or the entry of a medical device. Catheters may be used to introduce or remove fluids (including gases) from cavities, ducts, vessels, or other location in a patient's body. Catheters may be introduced into the body through any natural or surgically-created opening by means of a guidewire, sheath, stylet, trocar, etc.

One particular type of catheter, commonly referred to as a suction or drainage catheter, is commonly used to remove biological or manmade materials from a patient's body. To remove such materials, the catheter has appropriately-sized suction holes disposed along its body to fluidically couple the catheter lumen with an external environment. The proximal end of the catheter is typically coupled to a negative pressure source such as a vacuum pump. Examples of biological materials in the patient's body may include blood, urine, pus or substances secreted or produced by the patient's organs, severed or detached tissue, bodily gases, etc. Examples of manmade materials that may be removed from a patient's body include, but are not limited to, fluids introduced into the body or otherwise produced by a procedure, medicament and medical apparatus.

### SUMMARY

According to one aspect of the present invention, there is provided a catheter for suctioning materials from a location inside a patient's body, the catheter comprising an elongate body having adjacent longitudinally-extending suction and vent lumens separated by a dividing septum, a proximal end of the suction and vent lumens configured to be fluidically coupled to a source of negative pressure and a source of at least neutral vent pressure, respectively, suction holes in an exterior surface of the catheter each fluidically coupling the suction lumen with an exterior environment of the catheter, and at least one port through the septum that fluidically couples the suction and vent lumens, wherein the ratio of the area of the suction holes and ports is such that suction force at unobstructed suction holes is maintained in a desired range for a given negative pressure regardless of whether none, one or more than one suction hole is obstructed.

In another aspect of the present invention, there is provided a catheter suction system comprising a source of negative pressure; and a catheter, coupled to the source of negative pressure, configured to suction materials from a location inside a patient's body comprising: an elongate body having adjacent longitudinally-extending suction and vent lumens separated by a dividing septum, a proximal end of the suction and vent lumens configured to be fluidically coupled to a source of negative pressure and a source of at least neutral vent pressure, respectively, suction holes in an exterior surface of the catheter each fluidically coupling the suction lumen with an exterior environment of the catheter, and at least one port through the septum that fluidically couples the suction and vent lumens, wherein the ratio of the area of the suction holes and ports is such that suction force at unobstructed suction holes is maintained in a desired range for a given negative pressure regardless of whether none, one or more than one suction hole is obstructed.

In a third aspect of the present invention, there is provided a system for cryogenic spray ablation comprising a cryogen source; a catheter, connected to the cryogen source, configured to deliver the released cryogen onto target tissue of the patient; a source of negative pressure; and a catheter, coupled to the source of negative pressure, configured to suction materials from a location inside a patient's body comprising: an elongate body having adjacent longitudinally-extending suction and vent lumens separated by a dividing septum, a proximal end of the suction and vent lumens configured to be fluidically coupled to a source of negative pressure and a source of at least neutral vent pressure, respectively, suction holes in an exterior surface of the catheter each fluidically coupling the suction lumen with an exterior environment of the catheter, and at least one port through the septum that fluidically couples the suction and vent lumens, wherein the ratio of the area of the suction holes and ports is such that suction force at unobstructed suction holes is maintained in a desired range for a given negative pressure regardless of whether none, one or more than one suction hole is obstructed.

### BRIEF DESCRIPTION OF THE DRAWINGS

Embodiments of the present invention are described herein in conjunction with the accompanying drawings, in which:

FIG. 1 schematic view of an exemplary cryoablation system in which embodiments of the multi-lumen catheter of the present invention may be advantageously implemented;

FIG. 2 is a schematic view of one embodiment of a suction catheter system in accordance with one embodiment of the present invention;

FIG. 3A is a schematic cross-sectional view of a distal portion of one embodiment of the multi-lumen suction catheter illustrated in FIG. 2, during operation of the catheter system when no suction holes are obstructed;

FIG. 3B is a schematic cross-sectional view of a distal portion of one embodiment of the multi-lumen suction catheter illustrated in FIG. 2, during operation of the catheter system when one suction hole is obstructed;

FIG. 3C is a schematic cross-sectional view of a distal portion of one embodiment of the multi-lumen suction catheter illustrated in FIG. 2, during operation of the catheter system when a plurality of suction holes are obstructed;

FIG. 3D is a schematic cross-sectional view of a distal portion of one embodiment of the multi-lumen suction catheter illustrated in FIG. 2, during operation of the catheter system when a plurality of suction holes and a plurality of vent holes are obstructed;

FIG. 4A is a side view of a multi-lumen catheter according to one embodiment of the present invention;

FIG. 4B is an enlarged side view of a distal end of the multi-lumen catheter illustrated in FIG. 4A;

FIG. 4C is a top view of the multi-lumen catheter illustrated in FIG. 4A;

FIG. 4D is an enlarged top view of a distal end of the multi-lumen catheter illustrated in FIG. 4C;

FIG. 4E is an enlarged top view of a proximal end of the multi-lumen catheter illustrated in FIG. 4C;

FIG. 4F is a cross-sectional view of the multi-lumen catheter illustrated in FIG. 4C taken along cross-sectional lines F-F; and

FIG. 4G is a cross-sectional view of the multi-lumen catheter illustrated in FIG. 4C taken along cross-sectional lines G-FG.

### DETAILED DESCRIPTION

Aspects of the present invention are generally directed to a catheter having a plurality of longitudinal lumens for removing biological, natural and/or man-made materials from cavities, ducts, vessels, or other locations in a patient's body. The multi-lumen catheter comprises a longitudinally-extending suction lumen with a plurality of suction holes through which materials pass into the lumen in response to suction forces generated by a source of negative pressure coupled to a proximal end of the lumen. The catheter also comprises a longitudinally-extending vent lumen coupled to a source of at least neutral vent pressure through, for example, an opening to ambient air at the proximal end of the catheter, and preferably, through one or more vent holes disposed along a length of the catheter. A dividing septum between the adjacent lumens has one or more ports fluidically coupling the lumens. The ratio of the area of the suction holes and ports is such that the suction force at unobstructed suction holes is maintained below a desired maximum force for a given negative pressure when none or more of the suctions holes are obstructed.

Advantageously, the ports in the septum which fluidically couple the suction and vent lumens, sometimes referred to herein as communication ports, provide an alternative flow paths should a suction hole become obstructed by, for example, materials or internal tissue. When a suction hole obstruction occurs, fluid is drawn into the suction lumen through the communication port(s). In embodiments having a plurality of communication ports, a greater compensating flow occurs through those ports that are proximate to the obstructed suction hole. Fluid is drawn into the vent lumen via the opening to ambient air and/or through the vent holes. As with the communication ports, a greater compensating flow into the vent lumen occurs through those vent holes that are proximate to the obstructed suction hole. This compensating fluid flow prevents the suction forces from exceeding a predetermined maximum value during use even when one or more suction holes become obstructed. This maximum force may be set, for example, to avoid hematoma, to permit repositioning of the catheter during use, to avoid drawing in unwanted materials such as materials not proximate to the catheter, materials having greater than a certain mass, etc.

Embodiments of the present invention may be configured to be employed in a variety of surgical, diagnostic, preventative and other surgical and non-surgical procedures and treatments of a patient in which a biological, natural or manmade material is to be removed from the patient's body. As one of ordinary skill in the art will find apparent, such treatment sites may be, for example, the brain, esophagus, lungs, abdomen, heart, stomach, rectum, intestines, or other organ or anatomical feature of the body. Furthermore, embodiments of the multi-lumen catheter may be configured to be used in conjunction with the administration of fluids such as medication, saline, etc.

One exemplary application of a surgical procedure in which embodiments of the multi-lumen catheter of the present invention may be implemented to remove materials from a patient's body is cryosurgery or cryoablation (collectively and generally referred to as “cryosurgery” herein). Cryosurgery is a procedure in which diseased, damaged or otherwise unwanted tissue is frozen using a cryogen such as liquid nitrogen. The tissue is frozen by spraying cryogen onto a target tissue causing the tissue to freeze followed by period of time in which apoptosis occurs.

During cryosurgery, the cryogen is normally removed from the treatment site to prevent non-target tissue from being exposed to the cryogen. It may also be necessary to remove from the patient's body the gaseous byproduct of cryosurgery to avoid undesirable side effects. This removal may be accomplished using a suction catheter that is attached to a source of negative pressure such as a vacuum pump.

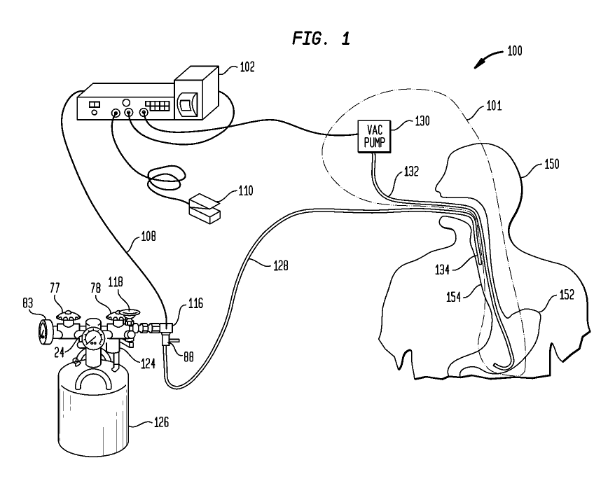

A simplified perspective view of an exemplary cryosurgery system is illustrated in FIG. 1. Cryosurgery system 100 comprises a pressurized cryogen storage tank 126 to store cryogen under pressure. In the following description, the cryogen stored in tank 126 is liquid nitrogen although cryogen may be other materials as described in detail below. A convenient size for tank 126 has been found to be a 5.5 liter size, although larger or smaller size tanks may be implemented depending on the particular application and operational environment. In one embodiment, tank 126 is a double-walled insulated tank with adequate insulation to maintain the liquid nitrogen at a very low temperature over a long period of time. In one embodiment, the pressure for the liquefied gas in tank is 22 psi. However, it is to be understood that tank 126 may maintain the liquid nitrogen or other cryogen at other pressures suitable for the particular application.

Tank 126 is equipped with a pressure building coil or tube 124 for maintaining pressure. This tube 124 comprises metal tubing running from the inside of tank 126 to the outside of tank 126 and returning back to the inside of tank 126. Tube 124, in operation, contains circulating liquid nitrogen. If the pressure in tank 126 drops below acceptable levels, valve 118 to tube 124 may be opened to circulate gas outside of tank 126 through tube 124. The liquid nitrogen in tube 124 outside tank 126 will be warmed and returned to tank 126. This warmed nitrogen liquid will cause the head pressure in tank 126 to increase, thereby allowing for more rapid delivery of liquid nitrogen to a cryogen delivery catheter 128. In the tube arrangement shown, valve 118 is hand operated, however, valve 118 could be automatically controlled. In such an embodiment, valve 118 may be controlled to start circulating liquid through tube 124 or a coil once the pressure in tank 126 drops to unacceptable levels, and to stop circulating once the pressure returns to an acceptable level. With normal pressure maintained in tank 126, liquefied gas will be more rapidly expelled from tank 126 to catheter 128. The force of gas expelled from tank 126 is a function of the temperature and pressure of the liquid nitrogen in tank 126. Because of the large temperature differential between the ambient temperature and the temperature of liquid nitrogen, only a short length of tube 124 is required.

Tank 126 is also equipped with other valves and gauges. A head gas valve 77 relieves head pressure, while a delivery solenoid valve 78 allows liquid nitrogen to flow to catheter 128 through controllable valve 116. Safety relief valves (not shown) on tank 126 are configured to relieve tank 126 of excessive tank pressure. For example, in one embodiment, two safety relief valves are implemented; one valve may open at 22 psi and the other valve may open at 35 psi. In addition, tank 126 is equipped with a head pressure gauge 83 and a liquid level gauge 84.

In this exemplary cryosurgery system, a foot pedal 110 is implemented to allow operator actuation of controllable valve 116. Foot pedal 110 has the advantage of allowing the physician's hands to be free during cryosurgery. Tank 126, heating tube 124, and foot pedal 110 collectively allow for quick delivery of adequate amounts for cryogenic spray to tissue requiring cryoablation.

In certain embodiments, cryosurgery system 100 forces super-cooled nitrogen gas through catheter 128 at low pressure. This is accomplished with an auxiliary pressure bleeder 88 positioned between tank 126 and catheter 128. Bleeder 88 eliminates the elevated pressure produced at catheter 128 caused by the reduced internal diameter of catheter 128 relative to the larger internal diameter of the tube supplying nitrogen gas to catheter 128; and by the volatilization of the liquid nitrogen to gas phase nitrogen. Bleeder 88 reduces such pressure by venting gas phase nitrogen out of bleeder 88. With this venting of gas phase nitrogen, liquid phase nitrogen exits the distal end of catheter 128 as a mist or spray at a pressure of approximately 35 psi compared with the tank pressure of approximately 22 psi. It is to be understood that bleeder 88 is used in this exemplary embodiment, but that other embodiments of the cryosurgery system do not require bleeder 88.

In the embodiment illustrated in FIG. 1, a conventional therapeutic endoscope 134 is used to deliver the nitrogen gas to target tissue within the patient. Endoscope 134 may be of any size, although a smaller diagnostic endoscope is preferably used from the standpoint of patient comfort. In certain embodiments, a specially designed endoscope having a camera integrated therein may also be used. As is known, an image received at the lens on the distal end of the camera integrated into endoscope 134 may be transferred via fiber optics to a monitoring camera which sends video signals via a cable to a conventional monitor or microscope, where the procedure can be visualized. By virtue of this visualization, the surgeon is able to perform the cryosurgery at treatment site 154.

As the liquid nitrogen travels from tank 126 to the proximal end of cryogen delivery catheter 128, the liquid is warmed and starts to boil, resulting in cool gas emerging from the distal end or tip of catheter 128. The amount of boiling in catheter 128 depends on the mass and thermal capacity of catheter 128. Since catheter 128 is of small diameter and mass, the amount of boiling is not great. (The catheter would preferably be “French Seven”.) When the liquid nitrogen undergoes phase change from liquid to gaseous nitrogen, additional pressure is created throughout the length of catheter 128. This is especially true at the solenoid/catheter junction, where the diameter of the supply tube relative to the lumen of catheter 128 decreases from approximately 0.5 inches to approximately 0.062 inches, respectively. In order to force low pressure liquid/gas nitrogen through this narrow opening, either the pressure of the supplied nitrogen must decrease or the diameter of catheter 128 must increase. Due to the fact that system 100 is not a highly pressurized system, a bleeder 88 may be implemented to solve this problem. Bleeder 88 is configured to allow the liquid phase nitrogen to pass through the reduced diameter catheter 128 without requiring modification of tank pressure or catheter diameter. Without a pressure bleeder 88, the pressure of gas leaving the distal end of catheter 128 would be too high and have the potential for injuring the tissue of the patient.

When the liquid nitrogen reaches the distal end of catheter 128 it is sprayed out of cryogen delivery catheter 128 onto the target tissue. It should be appreciated that certain embodiments the cryosurgery system may be able to sufficiently freeze the target tissue without actual liquid nitrogen being sprayed from catheter 128. In particular, a spray of liquid may not be needed if cold nitrogen gas is capable of freezing the target tissue.

Freezing of the target tissue is apparent to the physician by the acquisition of a white color, referred to as cryofrost, by the target tissue. The white color, resulting from surface frost, indicates mucosal freezing sufficient to destroy the diseased tissue. In one embodiment, the composition of catheter 128 or the degree of insulating capacity thereof will be selected so as to allow the freezing of the mucosal tissue to be slow enough to allow the physician to observe the degree of freezing and to stop the spray as soon as the surface achieves the desired whiteness of color. The operator may monitor the target tissue to determine when cryofrost has occurred via the camera integrated into endoscope 134. The operator manipulates suction catheter tube 132 and/or cryogen delivery catheter 128 to freeze the target tissue. Once the operation is complete, suction catheter 132, catheter 128, and endoscope 134 are withdrawn.

Because the invention uses liquid spray via catheter 128 rather than contact with a cold solid probe, the risk that an apparatus may stick to the tissue of the patient is reduced. Catheter 128 is further constructed and arranged so to reduce the potential for damage to the patient's tissue during the cryosurgery. For example, catheter 128 may comprise a plastic material having a low thermal conductivity and specific heat transfer properties, such as TEFLON, that reduces the potential that catheter 128 may stick to the tissue of the patient

Using cryogen delivery catheter 128 to deliver the cryogen permits a higher cooling rate (rate of heat removal) since the sprayed liquid evaporates directly on the tissue to which the cryogen is applied. The rate of re-warming of the target tissue is also high due to the fact that the applied liquid nitrogen boils away rapidly. No cold liquid or solid remains in contact with the tissue, and the depth of freezing is minimal.

Treatment site 154 as depicted in FIG. 1 is the esophagus of patient 150. It should be appreciated, however, that the treatment site but may be any location within patient 150 such as inside stomach 152 or other cavities, crevices, vessels, etc. Since freezing is accomplished by boiling liquid nitrogen, large volumes of this gas are generated. This gas must be allowed to escape. The local pressure will be higher than atmospheric pressure since the gas cannot easily flow out of the treatment site such as the gastrointestinal tract. In the illustrated embodiment, nitrogen gas will tend to enter stomach 152, which has a junction with the esophagus (the esophageal sphincter) immediately adjacent to treatment site 154. In this case, without adequate or quick suction, stomach 152 of patient 150 may become distended and become uncomfortable for patient 150. This buildup of gas could also potentially cause stomach 152 or its lining to become damaged or torn. As such, to prevent this buildup of gas in stomach 152, a suction tube 132 (e.g., a nasogastric tube) may be inserted into the patient to evacuate cryogen and other gases, particles, liquids, etc. from the patient. Suction may be provided by a suction pump 130 or other conventional source of negative pressure.

Also depicted in FIG. 1 is a control unit 102, which is connected to foot pedal 110, controllable valve 116 and pump 130. In this embodiment, an operator of cryosurgery system 100 may instruct control unit 102 to actuate controllable valve 116 via foot pedal 110. The operator may start the flow of cryogen by pressing on foot pedal 110, and may end the flow of cryogen by releasing foot pedal 110. The flow of cryogen may be fluctuated by exerting differing amounts of pressure on foot pedal 110. Actuation of foot pedal 110 causes control unit 102 controls controllable valve 116 via control line 108 to cause controllable valve 116 to open or close based on, for example, receiving operator inputs, thermal sensors (not shown) located at one or more points in system 100 or the environment outside system 100, pressure sensors (not shown), among others inputs. Although this illustrative embodiment describes the use of foot pedal 110 to enter user inputs it should be appreciated that other manners of entering operator inputs may be utilized, including buttons, switches, toggles, dials, user interfaces, etc. on, in, or coupled to control unit 102.

Suction catheter 132 and vacuum pump 130, collectively referred to herein as a catheter system 101, interoperate to remove biological, natural and/or man-made materials from cavities, ducts, vessels, or other locations in a patient's body. As described in detail below, catheter system 101 may incorporate any one of a myriad of embodiments of the multi-lumen catheter of the present invention as suction catheter 132.

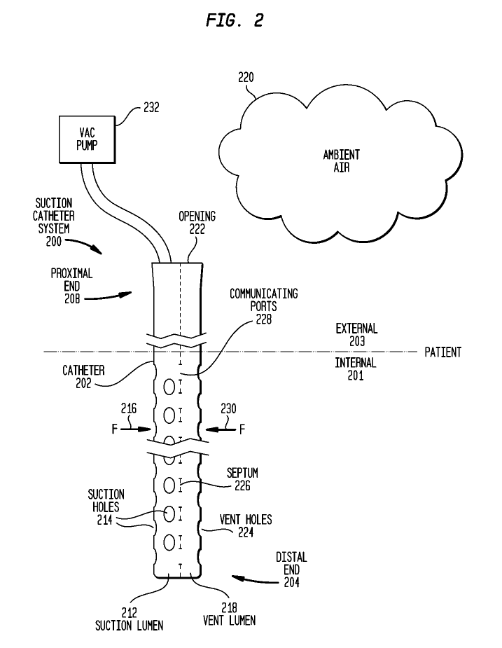

FIG. 2 is a simplified perspective view of one embodiment of catheter system 101, referred to herein as suction catheter system 200. As noted, catheter system 200 is configured to remove materials from cavities, ducts, vessels, or other locations in a patient's body. Catheter system 200 comprises an elongate catheter 202, referred to herein as multi-lumen suction catheter 202. Suction catheter 202 has a distal end 204 which, in this illustration, is positioned internal 201 to a patient. A proximal end 208 of catheter 202 is located external 203 to the patient.

As shown in this representative embodiment, multi-lumen catheter 202 has at least two longitudinally-extending lumens. Specifically, multi-lumen catheter 202 comprises a longitudinally-extending suction lumen 212 with a plurality of suction holes 214 through which materials pass into the lumen in response to suction forces 216 generated by a source of negative pressure 232 coupled to proximal end 208 of the lumen. Catheter 202 also comprises a longitudinally-extending vent lumen 218 fluidically coupled to a source 220 of at least neutral vent pressure (e.g., ambient air 220) through, for example, an opening 222 at catheter proximal end 208, and preferably, through one or more vent holes 224 disposed along a length of the catheter.

A dividing septum 226 between the adjacent lumens 212, 218 has at least one, and preferably a plurality, of communication ports 228 fluidically coupling lumens 212, 218. The ratio of the area of suction holes 214 and ports 228 is such that the suction force 216 at unobstructed suction holes 214 is maintained below a desired maximum force for a given negative pressure regardless of whether one or more suction holes 214 are partially or completely obstructed.

In this illustrative embodiment, vent holes 224A-224F, communication ports 228A-228F and suction holes 214A-214F are laterally aligned with each other. As one of ordinary skill in the art will appreciate, and as will be described in greater detail below, such lateral alignment, correspondence in quantity of vent holes 224, communication ports 228 and suction holes 212, similarity in size and dimension, etc., are illustrative only, and that such features of the multi-lumen catheter of the present invention may vary depending on the intended application.

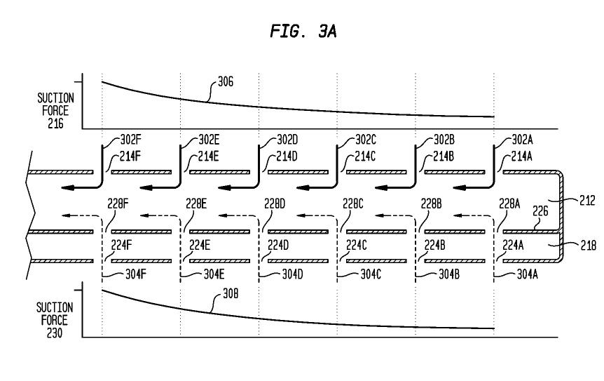

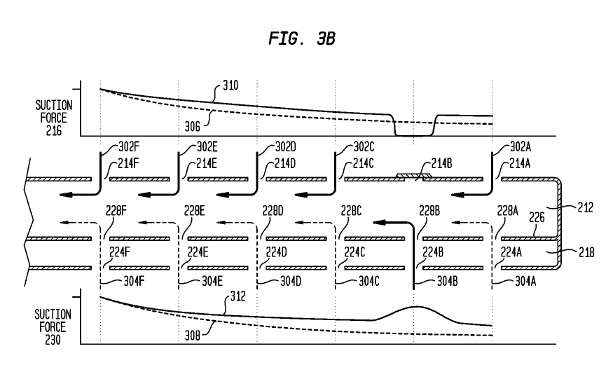

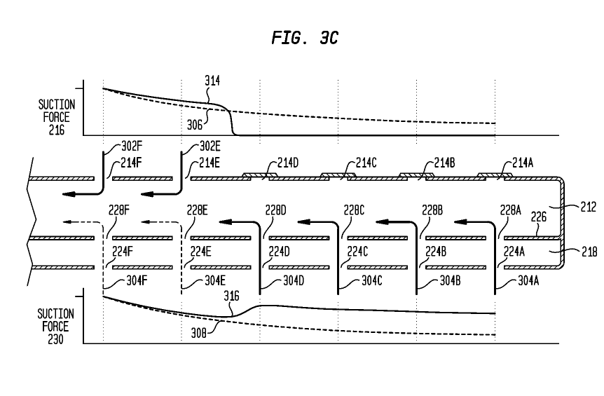

FIGS. 3A-3D are schematic cross-sectional views of a distal portion of multi-lumen suction catheter 202 illustrated in FIG. 2, during operation of catheter system 200. In FIG. 3A, no suction holes 214 are obstructed. In FIG. 3B, suction hole 214B is obstructed, in FIG. 3C, suction holes 214A through 214D are obstructed, and in FIG. 3D, suction holes 214A through 214F and vent holes 224A through 224F are obstructed.

Referring now to FIG. 3A, in response to a given negative pressure applied to the proximal end of suction lumen 212 (not shown), a suction force 216 is generated at each suction hole 214A-214F to draw fluid through the suction holes. Such flow is depicted by flow arrows 302A through 302F, respectively. There is also a suction force 230 generated at vent holes 224A-224F, resulting in a responsive fluid flow through the vent holes. This flow is depicted by flow arrows 304A through 304F. In this illustrative embodiment, suction force 216 is greater than suction force 230 due to, for example, the relative area of the suction holes, communicating ports and vent holes. The greater fluid flow 302 is depicted by solid flow arrows while the lesser fluid flow 304 is represented by dashed flow arrows.

As one of ordinary skill in the art will appreciate, suction force 216 at each successive suction hole 214, and hence the unobstructed fluid flow 302, decreases in proportion to the inverse square of the distance from the source of negative pressure 232. This is represented by graph 306 of suction force 216. Graph 306 is provided to illustrate the relative magnitude of suction force 216 at each suction hole 214. A similar relationship exists for communicating ports 228 and vent holes 224, as reflected in graph 308. Graph 308 represents the suction force at vent holes 304.

Because suction and vent lumens 212 and 218 are in fluid communication with each other via ports 228, and because vent lumen 218 is coupled to a source of at least neutral vent pressure through opening 222 (FIG. 2) and vent holes 224, vent lumen 218 provides an additional fluid flow paths into suction lumen 212. As noted, this additional flow path serves as an alternative flow path should a suction hole 214 become obstructed by, for example, materials or internal tissue. For example, in FIG. 3B a suction hole 214B is shown obstructed thereby preventing fluid flow through that hole. This is illustrated by the absence of fluid flow arrow 302B. This is also illustrated in graph 310 which illustrates a decrease of suction force 216 at obstructed suction hole 214B.

To compensate for this obstruction, fluid will be drawn into suction lumen 212 through communication ports 228 and vent holes 224. In this exemplary embodiment having a plurality of communication ports 228 and vent holes 224 each laterally adjacent to a suction hole 214, a greater compensating flow occurs through those ports 228 and vent holes 224 that are more proximate to obstructed suction hole 214B. This increased compensating flow is illustrated in graph 312, which shows suction force 230 increasing at vent hole 224B. Also, other ports 228 and vent holes 224 proximate to the obstructed suction hole 214B experience a relatively smaller increase in suction force in response to the increase at port 228B and vent hole 224B.

This is further illustrated in FIG. 3C in which a number of suction holes 214A-214D are obstructed. The suction force 216 at unobstructed suction holes 214E and 214F increases slightly due to the compensating effect of communicating vent lumen 218, while no suction force 216 is generated at obstructed suction holes 214A-214D. This is illustrated in graph 314. Similar to the scenario described with FIG. 3B, when suction holes 214A through 214D become obstructed, a greater compensating flow occurs through vent holes 304A through 304D. This is depicted by the solid flow arrows 304A through 304D, and the corresponding graph 316.

In addition to suction holes 214 becoming obstructed, vent holes 224 may also become obstructed, as illustrated in FIG. 3D. As shown in FIG. 3D, suction holes 214A through 214F and vent holes 224A through 224F are obstructed. At each of these obstructed holes, suction forces 216 and 230 decreases significantly, in certain situations of complete obstruction down to zero. The reduced suction forces 216 and 230 are illustrated by graphs 318 and 320, respectively. Since vent lumen 218 is fluidically coupled to source 220 (not shown) of at least neutral vent pressure (e.g., ambient air 220), a compensating flow occurs, originating from source 220, down through vent lumen 218, through port holes 228, and into suction lumen 212. This flow is depicted by solid flow arrows 322A through 322F. It is to be understood that where one or more vent holes 224 are obstructed, or where no vent holes 224 are obstructed, flow 332 may still occur to some extent simultaneously with flow 304.

Thus, embodiments of the present invention prevent suction forces 216 from exceeding a desired maximum value during use when one or more suction holes 214 become obstructed. This maximum force may be set, for example, to avoid hematoma, to permit repositioning of the catheter during use, to avoid drawing in unwanted materials such as those not proximate to the catheter, those having greater than a certain mass, etc.

Although suction holes 214, communicating ports 228 and vent holes 224 are shown in FIGS. 3A through 3C as being equal in size and spaced evenly along catheter 202, it is to be understood that the size and space of suction holes 214, communicating ports 228 and vent holes 224 may differ in other embodiments. For example, in one embodiment, in order to compensate for the inverse square law described above in conjunction with FIG. 3A, suction holes 214 may gradually decrease in size between successive holes 214 in the proximal direction. This configuration would allow greater suction force to be generated at the larger suction holes 214 than at the smaller suction holes 214. Communication ports 228 and vent holes 224 may be similarly sized differently with respect to one another in this or other embodiments to achieve the same or similar result.

In another embodiment, groups of suctions holes 214 may be equally sized, with each such group of suction holes 214 decreasing in size along catheter 202 in the proximal direction. Such a configuration would advantageously provide different suction force at each group, which can be adjusted to achieve a desired suction force for a given application.

Furthermore, in another embodiment, the space between each successive suction hole 214, or communication port 228 or vent hole 304, may increase in the proximal or distal direction. Such a configuration could provide, for example, more suction force to be exerted in areas of catheter 202 where the holes 214, 304 or ports 228 are closer together than in areas of catheter 202 where they are spaced further apart, thereby compensating for the inverse square effect described above.

Additionally, although suction holes 214, communication ports 228 and vent holes 304 are shown in FIGS. 3A through 3C as being aligned perpendicularly and in a 1-to-1-to-1 ratio, in other embodiments these holes 214, 304 and ports 228 may not be aligned and may be along catheter 202 in a ratio other than 1-to-1-to-1. For example, in one embodiment, there may only be half the number of ports 228 along catheter as the number of suction holes 214, while there may be three times as many ports 228 as there are vent holes 304 along catheter 202. Furthermore, suction holes 214, communication ports 228 and vent holes 304 may be arranged around the circumference of catheter and longitudinally spaced apart along catheter 202 so that holes 214, 304 and ports 228 are not aligned with respect to one another.

Furthermore, in another embodiment, for each region along catheter 202, suction holes 214 may generally be larger than communication ports 228 in the same region. For each of those catheter 202 regions, the size of suction holes 214 may be configured with respect to communication ports 228 to achieve various results when negative pressure is applied. For example, having suction holes 214 that are larger than communication ports 228 for a region of the catheter 202 may allow a greater suction force through suction hole 214 than through communication port 228.

Similarly, the size of communication ports 228 may be greater than the size of vent holes 304 for a given region of catheter 202. Where vent lumen 218 is coupled to ambient air 220, smaller vent holes 304 may result in more flow from ambient air 220 than from the areas immediately outside vent holes 304. The sizes of the various holes and ports as described above as well as the spacing between some or all of those holes and ports as well as their orientation and alignment along catheter 202 may be advantageously configured in different embodiments of the present invention.

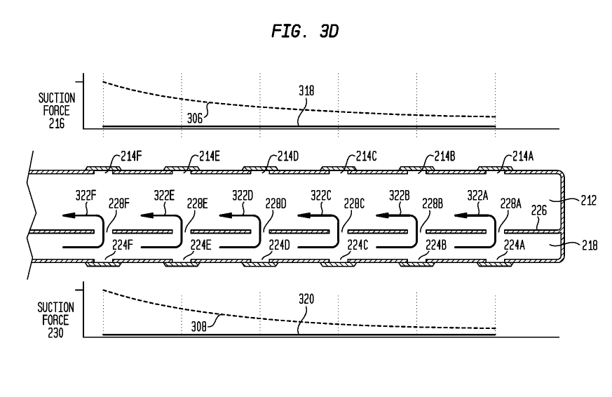

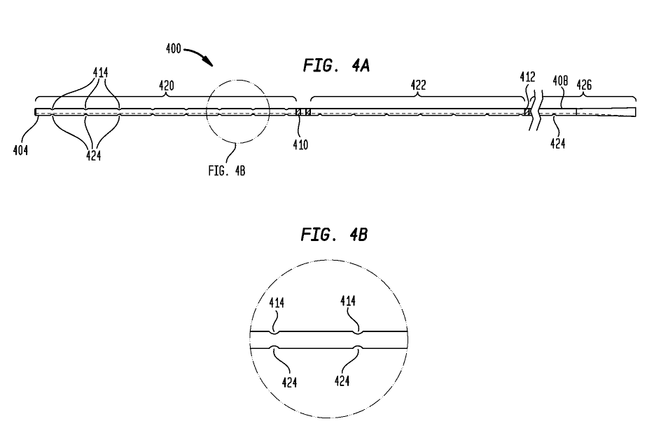

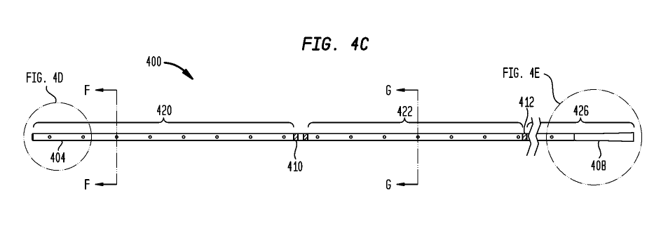

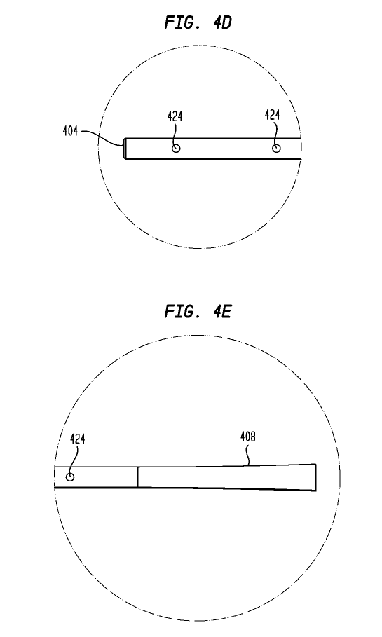

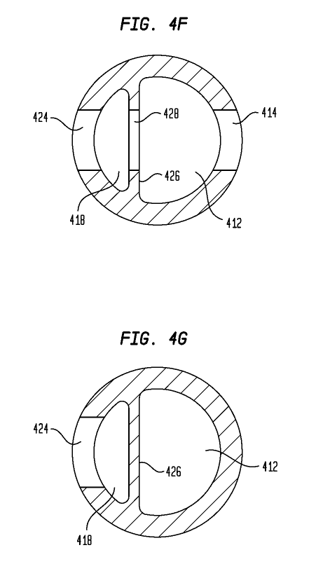

FIG. 4A is a side view of a multi-lumen catheter according to one embodiment, referred to herein as multi-lumen suction catheter 400. FIG. 4B is an enlarged view of a medial section of catheter 400. Suction catheter 400 is configured to be utilized as a cryogen delivery catheter 128 in a cryosurgery system such as system 100 described above with reference to FIG. 1.

As noted above, when treating a condition such as Barrett's esophagus with cryosurgery system 100, a large volume of nitrogen gas is formed from spraying liquid nitrogen onto the target tissue, which is typically proximate to the esophageal sphincter. Depending on the rate at which the gas is formed, the location of the treatment site and other factors, some or all of the nitrogen gas may travel up the esophagus to be discharged from the patient's body. Some of the nitrogen gas may also enter stomach 152 (FIG. 1) through the esophageal sphincter. In this case, without adequate or quick suction, stomach 152 may become distended and the patient may experience discomfort. This buildup of gas could also potentially cause stomach 152 or its lining to become damaged or torn.

As shown in FIG. 4A, multi-lumen suction catheter 400 is functionally divided into different longitudinal sections. The distal section, referred to as gastric section 420, is demarcated by gastric marker 410 and is configured to be placed in stomach 152. The next longitudinal section, referred to as esophageal section 422, is demarcated by gastric marker 410 and esophageal marker 412, as is configured to be placed in esophagus. The proximate longitudinal section 426 extends from the patient's body and is coupled to a source of negative pressure such as vacuum pump 130 (FIG. 1).

Suction holes 414 are disposed along the catheter, from distal end 404 to gastric marker 410. Vent holes 424 are disposed along catheter 400 on an opposite side of catheter 400 and are disposed along substantially the entire length of the catheter. In this particular embodiment, vent holes 424 are also provided in proximate longitudinal section 426.

Gastric marker 410 and esophageal marker 412 advantageously provide viewable marks that can be used by a surgeon during treatment. For example, in a surgery in which an endoscope is being used in conjunction with catheter 400, gastric marker 410 may be monitored on a display connected to the endoscope, and catheter 400 may be moved or otherwise manipulated using gastric marker 410 as a reference point to provide stronger or additional suction to various areas within the patient's esophagus or stomach. Esophageal marker 412 may be utilized for similar purposes.

FIG. 4C shows catheter 400 with distal end 404 and proximal end 408, aspects of which will be discussed further below in conjunction with FIGS. 4D and 4E, respectively. Cross-sectional area F-F and G-G will also be discussed further below in conjunction with FIGS. 4F and 4G, respectively.

In FIG. 4D, distal end 404 of catheter 400 is shown, along with vent holes 424. As can be seen, the end of catheter 400 has beveled or tapered edges which advantageously minimize the likelihood of injury as catheter 400 is inserted into a patient. Furthermore, distal end 404 of catheter 400 may be partially open to allow objects such as a guide wire to pass through catheter 400. In other embodiments, distal end 404 may be open so that the vent lumen and/or suction lumen may be in full communication with the external environment of catheter 400. In the embodiment in which both the vent lumen and the suction lumen have open distal ends, air may fluidically pass across the catheter tip from lumen to lumen.

In FIG. 4E, proximal end 408 of catheter 400 is tapered, where the tapered shape may serve a variety of purposes. For example, the tapered shape may have a larger internal diameter in the tapered portion of catheter 400 in order to accommodate a connector hose (not shown) that is coupled to negative pressure source 232, where the connector hose preferably has a similar internal diameter as the diameter of the suction lumen. Also, proximal end 408 of catheter 400 has an additional vent hole 224 that may be positioned in the patient's mouth or outside the patient's body to provide an additional hole through which ambient air may pass.

FIGS. 4F and 4G depict cross-sections F-F and G-G of catheter 400 shown in FIG. 4C. Cross-section F-F is taken in gastric section 420 while cross-section G-g is taken in esophageal section 422. Suction lumen 412 is shown as being larger than vent lumen 418 in the embodiment shown, although it is to be understood that vent lumen 418 may have an equal size or capacity as suction lumen 412 in other embodiments of the present invention. Vent hole 424 is shown as being disposed on one side of catheter 400 while suction hole 214 4s disposed on an opposing side of catheter 400. Communication port 428 is shown along septum 426 within catheter 400.

As shown in FIGS. 4A, 4F and 4G, vent holes 424 are provided in gastric section 420 and esophageal section 422, while suction holes 414 and communication ports 426 are provided only in gastric section 420. This is because the nitrogen gas that does not travel into stomach 152 is generally discharged from the patient without suction. In contrast, the nitrogen gas that travels into the stomach is not discharged naturally due to the esophageal sphincter and presence of the cryogen delivery catheter 128 and suction catheter 400.

Suction holes 414 in gastric section 420 are utilized to draw in such nitrogen gas. Should one or more suction holes 414 become obstructed from, for example, biological material such as mucous or due to catheter 400 being positioned against to the stomach wall, the suction force at the unobstructed suction holes 414 is maintained below a predetermined maximum force without interrupting the application of suction. This allows for continuous suction that, for example, prevents hematoma, permits repositioning of the catheter, etc. while ensuring that the nitrogen gas is quickly and effectively evacuated from the stomach.

Should vent holes 424 in gastric section 420 also become obstructed, vent holes 424 in other sections including esophageal section 422 and proximate section 426 provide the requisite air flow to enable the suction force at unobstructed suction holes 414 to be maintained below the desired maximum level. Similarly, should vent holes 424 in gastric section 420 and esophageal section 422 become obstructed, vent holes 424 and opening 222 (FIG. 2) in proximate section 426 will provide the requisite air flow to enable the suction force at unobstructed suction holes 414 to be maintained below the desired maximum level.

Embodiments of the present invention may be manufactured using various techniques. Catheters 202, 400 may be formed through extrusion, blow extrusion, injection moulding, blow moulding, rotational moulding, compression moulding, reaction injection moulding, vacuum moulding, fabrication, through the use of nanotechnology and materials formed through nanotechnology, weaving, stamping, weaving, and other method now known or later developed. Further methods may be used to form the various holes and ports according to the present invention, including but not limited to drilling, melting, burning, radiating, etc. Also, the multi-lumen catheter of the present invention may be integrally formed by joining two or more separately-manufactured catheters.

In some embodiments, a coating that enhances lubricity such as a hydrophilic coating may be provided within one or more lumens in embodiments of the multi-lumen catheter of the present invention. Such a hydrophilic coating may facilitate the guiding of the catheter down a pre-positioned guide wire as the catheter is inserted into the patient.

Although the present invention has been fully described in conjunction with several embodiments thereof with reference to the accompanying drawings, it is to be understood that various changes and modifications may be apparent to those skilled in the art. For example, in the embodiment described above with reference to FIG. 4A, multi-lumen suction catheter 400 is functionally divided into different longitudinal sections demarcated by markers. It should be appreciated that in alternative embodiments such longitudinal sections may include more or less than that described herein, with the sections of the catheter having the same or different configurations, hole and port configuration, etc. It should also be appreciated that the use of markers in such embodiments of the catheter may be the same or different and may be fixed or adjustable, etc. Such changes and modifications are to be understood as included within the scope of the present invention as defined by the appended claims, unless they depart therefrom.

What is claimed is:

1. A catheter configured to suction materials from a location inside a patient's body comprising: an elongate body having adjacent longitudinally-extending suction and vent lumens separated by a dividing septum, a proximal end of the suction and vent lumens configured to be fluidically coupled to a source of negative pressure and a source of at least neutral vent pressure, respectively, suction holes in an exterior surface of the catheter each fluidically coupling the suction lumen with an exterior environment of the catheter, and at least one port through the septum that fluidically couples the suction and vent lumens, wherein the ratio of the area of the suction holes and ports is such that suction force at unobstructed suction holes is maintained in a desired range for a given negative pressure regardless of whether none, one or more than one suction hole is obstructed.

2. The catheter of claim 1, further comprising:
a plurality of vent holes in an exterior surface of the catheter each fluidically coupling the vent lumen with an exterior environment of the catheter.

3. The catheter of claim 1, wherein the suction holes, vent holes and communicating ports are laterally aligned with each other.

4. The catheter of claim 1, further comprising:
a material coating the surface of one of said lumens to enhance lubricity.

5. The catheter of claim 1, wherein the catheter comprises:
contiguous, longitudinal sections each configured to be inserted into a different region in the patient, and each having a different arrangement of two or more of the suction holes, vent holes and communicating ports.

6. A catheter suction system comprising:
a source of negative pressure; and
a catheter, coupled to the source of negative pressure, configured to suction materials from a location inside a patient's body comprising: an elongate body having adjacent longitudinally-extending suction and vent lumens separated by a dividing septum, a proximal end of the suction and vent lumens configured to be fluidically coupled to a source of negative pressure and a source of at least neutral vent pressure, respectively, suction holes in an exterior surface of the catheter each fluidically coupling the suction lumen with an exterior environment of the catheter, and at least one port through the septum that fluidically couples the suction and vent lumens, wherein the ratio of the area of the suction holes and ports is such that suction force at unobstructed suction holes is maintained in a desired range for a given negative pressure regardless of whether none, one or more than one suction hole is obstructed.

7. The catheter suction system of claim 6, further comprising:
a plurality of vent holes in an exterior surface of the catheter each fluidically coupling the vent lumen with an exterior environment of the catheter.

8. The catheter suction system of claim 6, wherein the suction holes, vent holes and communicating ports are laterally aligned with each other.

9. The catheter suction system of claim 6, further comprising:
a hydrophilic material coating the surface of one of said lumens.

10. The catheter suction system of claim 6, wherein the catheter comprises:
contiguous, longitudinal sections each configured to be inserted into a different region in the patient, and each having a different arrangement of two or more of the suction holes, vent holes and communicating ports.

11. A system for cryogenic spray ablation comprising:
a cryogen source;
a catheter, connected to the cryogen source, configured to deliver the released cryogen onto target tissue of the patient;
a source of negative pressure; and
a catheter, coupled to the source of negative pressure, configured to suction materials from a location inside a patient's body comprising: an elongate body having adjacent longitudinally-extending suction and vent lumens separated by a dividing septum, a proximal end of the suction and vent lumens configured to be fluidically coupled to a source of negative pressure and a source of at least neutral vent pressure, respectively, suction holes in an exterior surface of the catheter each fluidically coupling the suction lumen with an exterior environment of the catheter, and at least one port through the septum that fluidically couples the suction and vent lumens, wherein the ratio of the area of the suction holes and ports is such that suction force at unobstructed suction holes is maintained in a desired range for a given negative pressure regardless of whether none, one or more than one suction hole is obstructed.

12. The catheter suction system of claim 11, further comprising:
a plurality of vent holes in an exterior surface of the catheter each fluidically coupling the vent lumen with an exterior environment of the catheter.

13. The catheter suction system of claim 11, wherein the suction holes, vent holes and communicating ports are laterally aligned with each other.

14. The catheter suction system of claim 11, further comprising:
a hydrophilic material coating the surface of one of said lumens.

15. The catheter suction system of claim 11, wherein the catheter comprises:
contiguous, longitudinal sections each configured to be inserted into a different region in the patient, and each having a different arrangement of two or more of the suction holes, vent holes and communicating ports.
